(19) United States
(12) Patent Application Publication
(10) Pub. No.: US 2010/0167328 A1
(43) Pub. Date: Jul. 1, 2010

(54) BLOOD CELL SEPARATION

(76) Inventors: Neil David Avent, Bristol (GB); Zoe Eileen Plummer, Bristol (GB); David John Head, Bristol (GB)

Correspondence Address: BANNER & WITCOFF, LTD., 1100 13th STREET, N.W., SUITE 1200, WASHINGTON, DC, 20005-4051, US

(21) Appl. No.: 12/376,995

(22) PCT Filed: Aug. 10, 2007
(86) PCT No.: PCT/GB07/03066

§ 371 Date: Jun. 1, 2009

(30) Foreign Application Priority Data
Aug. 11, 2006 (GB) 0616045.1

(51) Int. Cl. C12Q 1/26; C12Q 1/02; C12N 5/00; C12N 5/02; C12M 1/34

(52) U.S. Cl. 435/25; 435/29; 435/325; 435/375; 435/287.1

(57) ABSTRACT

There is provided a method of isolating foetal cells from an isolated sample of maternal blood, the method comprising identifying cells having a different expression pattern of at least one foetal marker compared to the expression pattern of the marker in an equivalent maternal cell and selecting the identified cells, characterised in that the foetal marker is selected from: HSP-60, a monoamine oxidase, glutamine synthase, Ara-70, Ara-54, FLJ20202, DCN-I protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, Peroxiredoxin 2. There is also provided a method of cultivating foetal cells and a foetal cell isolation kit.

### FIELD OF THE INVENTION

This invention relates to the field of prenatal diagnosis and, in particular, to the separation of foetal cells from maternal peripheral blood. Specifically, the invention relates to methods of separation of foetal cells from maternal blood cells and to apparatus for separating foetal blood cells from maternal blood cells.

### BACKGROUND

Current methods of prenatal diagnosis of disease involve invasive techniques. For example, such techniques include amniocentesis, chorionic villus sampling and cordocentesis. Millions of such analyses are currently performed every year using invasively sampled material for detecting chromosome abnormalities such as Down syndrome and other genetically inherited conditions. In the United States alone, approximately 200,000 invasive prenatal testing procedures such as amniocentesis and chorionic villus sampling are performed each year. Such tests are generally carried out on women over 35 and those with other risk factors, but most children with chromosomal or genetic defects are still born to women under the age of 35. These genetic disorders currently can be detected only by use of material obtained during invasive procedures. In England and Wales there have been, on average, around 630,000 live births per year for the last 10 years, but the average maternal age has risen from 28.5 years in 1995 to 29.5 years in 2005. The likelihood of a foetus carrying a genetic abnormality increases massively with maternal age and it is expected that this age will continue to increase. Invasive prenatal diagnosis is generally accepted as being risky to mother and foetus, with 1-2% of all procedures resulting in spontaneous miscarriage of the foetus.

It is known to be desirable to provide non-invasive alternatives to current methods for prenatal diagnosis of disease. It is hoped that non-invasive prenatal diagnostic techniques will eliminate or reduce the risks outlined above and will allow the expansion of prenatal testing in general. Non-invasive prenatal diagnosis using isolated foetal cells would also be more economical (i.e. not requiring a surgical procedure) than amniocentesis and chorionic villus sampling.

It is known that the blood plasma of pregnant women contains both foetal and maternal circulatory extracellular DNA and RNA. It is known to separate such foetal and maternal DNA on the basis of size differences between the two types of DNA, allowing enrichment of foetal material (see, for example, EP-A-1524321). However, the use of circulatory foetal nucleic acid is currently only used in the detection of paternally-inherited alleles as a result of the high levels of free maternal circulatory DNA. Foetal DNA, specifically polymorphic forms of placentally encoded species, have been used for prenatal Down syndrome diagnosis.

It has been known for decades that foetal cells are found in the peripheral blood of all pregnant women. As such, they represent an important potential target for non-invasive prenatal diagnosis, since most of these foetal cells are nucleated. The foetal cell types which have been identified in maternal blood include erythroblasts (nucleated red blood cells), lymphocytes, mesenchymal stem cells and placentally derived trophoblasts. If these cells could be isolated to homogeneity (i.e., devoid of contaminating maternal cells) genetic testing could be performed on the isolated cells. This would enable routine and safe non-invasive genetic testing for such disorders as aneuploidy, cystic fibrosis, beta thalassaemia and other inherited single gene disorders.

However, studies (e.g., Hahn, S et al., Molecular Human Reproduction (1998) 4 515-521) to assess the viability of the non-invasive prenatal diagnosis using foetal cells from maternal blood using methods such as fluorescent in situ hybridisation (FISH), density gradient/flow activated cell sorting (FACS) and magnetic bead activated cell sorting (MACS) cell isolation technology have, unfortunately, not used unique foetal markers, but instead have used cell-surface markers found on some maternal cells (for example, the transferrin receptor, CD71 or glycophorin A CD235a, see, e.g., WO96/09409). Furthermore, attempts have been made to isolate foetal erythroblasts using the internal foetal-specific globins epsilon and gamma, but since the proteins are also rarely expressed in adult cells (so-called “F-cells”) and exploitation causes destruction of the foetal cell, their uses are limited. Currently, the technical approach utilised to isolate foetal erythroblasts utilises such markers as glycophorin A which are, in fact, expressed equally on maternal and foetal erythroid cells (e.g. Al Mufti et al. (2004) Clin. Lab. Hematol. 26 123-128).

There are no specific details in the literature of significant biochemical differences between foetal and adult erythroid cells except that known for the epsilon and gamma globins and the Ii blood group of antigens, of which i is foetal specific.

There is, therefore, a need to provide true foetal cell specific markers.

International patent application WO 2004/078999 discloses a method of isolating foetal cells from maternal blood using a marker specific for the foetal cell. The method comprises identifying an allele encoding an antigen which is present in the DNA of the foetal cell but absent from maternal DNA, binding to the foetal cell an affinity reagent which recognises the antigen and selecting cells by the affinity reagent. The preferred antigen is a cell surface protein, particularly a human lymphocyte antigen (HLA) protein. However, there are disadvantages to this approach. For example, the system requires the determination of the HLA type of the father of the foetus (notoriously unreliable when considering cases of doubtful parentage) and the results may not be clearly reproducible.

There is also a need for efficient methods for cultivating foetal cells isolated from maternal blood.

It is known to separate foetal cells from maternal blood using physical separation techniques, e.g., see WO00/060351, which relates to density gradient centrifugation. However, current procedures based on density gradients may alter cells physiologically (Hahn, S et al. Molecular Human Reproduction (1998) 4 515-521). This may include the onset of apoptosis, signs of which (judged by nuclear condensation) were seen in a significant proportion of erythroblasts isolated from maternal blood in a recent study (Babochkina, et al. Haematologica (2005) 90 740-745). Alternatively, foetal cells can be obtained from and enriched from cervical canal aspirates by a combination of density gradient separation and antibody-mediated selection, for example, as disclosed in WO 2004/076653.

Hohmann et al., (Fetal Diagn. Ther. (2001) 16 52-56) assesses the use of various antibodies to detect foetal-originating cells.

US-A-2006/0105353 and Bianchi et al. (Prenatal Diagnosis (1996) 16 289-298) disclose methods of separating foetal cells by using CD45 antibodies and CD71 antibodies, with WO94/25873 disclosing a separation method using CD45 antibodies.

### SUMMARY OF THE INVENTION

According to a first aspect of the invention, there is provided a method of isolating foetal cells from a sample of maternal blood (which is preferably isolated), the method comprising identifying cells having a different expression pattern of at least one foetal marker (preferably 1, 2, 3, 4 or 5 markers) compared to the expression pattern of the marker in an equivalent maternal cell and selecting the identified cells, characterised in that the foetal marker is selected from: HSP-60 (Heat Shock Protein 60, GenBank accession no. P10809), a monoamine oxidase, glutamine synthase (accession no. P15104), Ara-70 (Androgen Receptor Associated Protein 70, accession no. Q13772), Ara-54 (Androgen Receptor Associated Protein 54, accession no. Q9UBS8), human hypothetical proteins MGC10526 (accession no. Q5JSZ7) or MGC10233 (accession no. NP_{—}689928), FLJ20202 (HGNC FAM46C), DCN-1 protein (accession no. NM_{—}020640), RAB5A (accession no. P20339, also known as HCC-10, Cervical Cancer oncogene 10 protein), HSP-7C (Heat shock cognate 71 kDa protein, accession no. P11142), EF1A1 (elongation factor 1-alpha 1, accession no. P68104), GRP78 (78 kDa glucose-regulated protein [precursor] GRP 78, accession no. P11021), MYL4 (myosin light polypeptide 4 myosin light chain 1, accession no. P12829), DnaJ homolog subfamily B member 14 (accession no. Q8TBM8), Vinculin (accession no. P18206), Desmoplakin (accession no. P15924), AMMECR1-like protein (accession no. Q6DCA0), Extracellular matrix protein 2 precursor protein (accession no. O94769), uncharacterised protein Cxorf57 (accession no. Q6NS14), Peroxiredoxin 1 (accession no. Q06830), Peroxiredoxin 2 (accession no. P32119). Preferably, the method further comprises separating the identified cells from other cells not having the different expression pattern of the at least one foetal marker.

The term “different expression pattern”, as used throughout this specification, indicates that the expression of a marker in a foetal cell is different to the expression of that marker in an equivalent maternal cell, i.e. in the same cell type derived from the mother (for example, erythroid cells such as erythroblasts). The comparison in marker expression is to be made between like-for-like cells from mother and foetus, e.g., the expression pattern in foetal erythroblasts compared with the expression pattern in maternal erythroblasts.

The difference in the expression pattern may be, for example, in the localisation of the marker to a particular cellular compartment (such as to the cell membrane) in a foetal cell but not in an equivalent maternal cell; an increased or decreased amount of a marker protein in the total protein of a foetal cell compared to that of an equivalent maternal cell; or expression of a marker in a foetal cell but not in an equivalent maternal cell. It may also relate to an increase or decrease in activity of a particular biochemical pathway in which the said protein species is actively involved.

The expression pattern in a given cell may be measured by standard molecular biology techniques such as those described in this specification, for example by determining the amount of mRNA in a cell, or by assaying the amount of a given protein present in a cell or in a cell compartment such as the cell surface membrane.

The term “an increased amount”, as used throughout this specification, indicates that the amount of a foetal marker expressed in a cell of interest, e.g. a foetal-derived erythroid cell, is greater than the amount of the foetal marker expressed in a cell which is not of interest, i.e. a maternal-derived cell. Preferably, cells which are not expressing an increased amount (i.e., cells expressing a significantly lower amount compared to foetal-derived cells) of each at least one foetal marker are maternal cells.

The term “a decreased amount”, as used throughout this specification, indicates that the amount of a foetal marker expressed in a cell of interest, e.g. a foetal-derived erythroid cell, is less than the amount of the foetal marker expressed in a cell which is not of interest, i.e. a maternal-derived cell. Preferably, cells which are not expressing an decreased amount (i.e., cells expressing a significantly higher amount compared to foetal-derived cells) of each at least one foetal marker are maternal cells. Such biomarkers, found to be upregulated on adult (maternal) erythroid cells compared to foetal erythroid cells, permits the elimination of maternal cells from mixtures of foetal and maternal erythroid cells.

In a preferred embodiment, the method of the invention comprises identifying cells expressing at least one foetal marker on the cell surface and selecting those cells. The foetal marker may be HSP-60, GRP 78, HSP-7C, MYL4 or EF1A1 and, preferably, is HSP-60. Preferably, the method further comprises separating the identified cells from cells not expressing the foetal marker on the cell surface.

The heat shock proteins are a family of highly conserved protective (chaperone) proteins and their expression is known to be induced by a range of stresses such as heat shock, exposure to heavy metals, toxins such as ethanol, exposure to UV light, infection, starvation, dehydration and hypoxia. HSP-60 cell surface expression, in particular, responds to exposure of a cell to hypoxia (low oxygen environment, to which foetal erythroid cells are known to be exposed) by translocation of the protein from mitochondria to the plasma membrane of the cell. HSP-60 decreases upon re-oxygenation and is reported to be expressed in human placenta. More interestingly, mice devoid of HSP-60 have been shown to be incapable of embryonic development showing the importance of this protein during the development of at least this species. Autologous HSP-60 acts as a danger signal for the innate immune system and its translocation of the protein to the membrane of cells such as lymphocytes and monocytes is associated with disease or responses to stress (Pfister et al. (2005) J. Cell. Sci. 118 1587-1594; Lang et al. (2005) J. Am. Soc. Nephrol. 16 383-391; Multhoff (2006) Handbook Exp. Pharmacol, 172 279-304; Romano et al. (2004) Int. Immunopharmacol. 4 1067-1073; Belles et al. (1999) Infect. Immun. 67 4191-4200). It is highly unlikely therefore that this protein will be present on the surface of the mature adult erythrocyte in healthy individuals, including pregnant women.

The inventors have uniquely and surprisingly discovered that foetal erythroid membranes contain HSP-60, which is completely absent from adult erythrocyte membranes. In normal circumstances, HSP-60 is localised to the mitochondria, but translocates to the cell surface during stress to the cell. An example of such stress is the anoxia under which foetal erythroid cells live. Immunisation to E. coli HSP-60 has been previously proposed for use in the treatment of rheumatoid arthritis (Bloemendal et al. (1997) Clin. Exp. Immunol. 110 72-78; WO 2006/032216).

Foetal cells or subpopulations thereof may be partially purified from maternal cells prior to the isolation process, for example, on the basis of expression of erythroid markers, for example by using density centrifugation followed by MACS/FACS and anti-glycophorin A or anti-Rh associated glycoprotein (RhAG), or using any biomarker specific for erythroid cells. This prior enrichment of erythroid lineage cells from maternal peripheral blood may greatly increase the efficacy of foetal cell isolation and enrichment, with the aim of reaching homogeneity.

Alternatively, the markers used in the method of the invention may allow a mixture of foetal erythroblasts and maternal non-erythroblast cells to be separated from maternal erythroblasts. Subsequently, foetal erythroblasts can be isolated from the mixture by use of erythroblast-specific markers, such as glycophorin A (GPA). Alternatively, the simultaneous separation of erythroid cells based on the presence of a known erythroid marker and a marker identified in this invention will lead to the isolation of pure foetal erythroid cells.

The selected foetal cells may be separated from the maternal blood or enriched by conventional separation techniques such as immunomagnetic (MACS) or other methods of cell sorting, for example FACS. Alternatively, the selected foetal cells may be separated or enriched by a physical binding agent, such as an affinity agent (antibody, aptamer or mimetic peptide). Suitable affinity agents include, without limitation, antibodies, Affibody molecules and domain antibodies. The affinity agent may be bound to a surface such as a bead. Preferably, the affinity agent is an antibody. Where the foetal specific marker is HSP-60, the antibody is preferably an anti-HSP-60 antibody or an aptamer reacting with HSP-60.

In an alternative or additional preferred embodiment, the method of the invention comprises identifying cells expressing a monoamine oxidase and selecting those cells. These foetal markers have unexpectedly been determined to be uniquely expressed in foetal cells but not maternal cells. Preferably, the method further comprises separating the identified cells from cells not expressing a monoamine oxidase.

In a further preferred embodiment of a method according to this aspect of the invention, cells are identified which express HSP-60 on the cell surface and:

an increased amount of at least one further foetal marker, the at least one further foetal marker being selected from: a monoamine oxidase, glutamine synthase, Ara-70, Ara-54, human hypothetical proteins MGC10526 or MGC10233, FLJ20202, DCN-1 protein, RAB5A, HSP-7C, EF1A1, GRP78, MYL4, DnaJ homolog subfamily B member 14, Vinculin, Desmoplakin, AMMECR1-like protein, uncharacterised protein Cxorf57; or a decreased amount of at least one further foetal marker being selected from: Extracellular matrix protein 2 precursor protein, Peroxiredoxin 1, Peroxiredoxin 2.

The identification of expression or of increased or decreased expression of the various markers may be simultaneous for all markers.

In an additional or alternative preferred embodiment of a method according to this aspect of the invention, cells are identified which express a monoamine oxidase and:

an increased amount of at least one further foetal marker, the at least one further foetal marker being selected from: HSP-60, glutamine synthase, Ara-70, Ara-54, human hypothetical proteins MGC10526 or MGC10233, FLJ20202, DCN-1 protein, RAB5A, HSP-7C, EF1A1, GRP78, MYL4, DnaJ homolog subfamily B member 14, Vinculin, Desmoplakin, AMMECR1-like protein, uncharacterised protein Cxorf57; or
a decreased amount of at least one further foetal marker being selected from: Extracellular matrix protein 2 precursor protein, Peroxiredoxin 1, Peroxiredoxin 2.

The identification of expression or of increased or decreased expression of the various markers may be simultaneous for all markers.

Alternatively, in a further preferred embodiment of the invention, any combination of two or more foetal markers may be used in the method, each of the two or more markers being selected from: HSP-60, a monoamine oxidase, glutamine synthase, Ara-70, Ara-54, human hypothetical proteins MGC10526 or MGC10233, FLJ20202, DCN-1 protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein. Preferably, at least one of the foetal markers is HSP-60, or a monoamine oxidase. The markers may be used in simultaneous or separate combination.

Therefore, the foetal cells may be initially isolated using a first foetal marker and then further separated or enriched on the basis of another foetal marker. The first marker may be a marker, such as a protein, which is expressed on the surface of foetal cells but not on the surface of maternal cells, e.g. HSP-60, or which is expressed in foetal cells but not in maternal cells, e.g. a monoamine oxidase. Preferably, the first marker is one which is located on the cell surface, e.g. HSP-60. The further foetal marker might, for example, be expression of an enzyme, e.g. a monoamine oxidase.

These markers used in the method according to the invention can, therefore, advantageously be used to separate foetal cells from maternal cells using a procedure which is non-invasive to the foetus, the maternal blood having been isolated from the mother prior to any manipulation of the foetal cells. The foetal cells may then be used to detect possible diseases in the foetus from which the cells are ultimately derived, such as Down syndrome and other aneuploidies, spina bifida, cystic fibrosis, beta thalassaemia and other genetically inherited conditions.

Where the marker is an enzyme which can convert supplied substrates into detectable products, fluorescent markers/probes may preferably be employed in order to allow visualisation of substrate metabolism products produced within the foetal cells only. Such a technique would allow a FACS-based approach to be utilised in separation of foetal and maternal blood cells.

Preferably, the foetal marker or further foetal marker is a monoamine oxidase, more preferably MAOA (accession no. NP_{—}000231) or MAOB (accession no. AAB27229). These enzymes both catalyse the oxidative deamination of bioactive amines (for example serotonin, epinephrine and norepinephrine) and thus may serve to protect the foetus from the movement of these bioactive amines across the placenta from the maternal circulation.

In an alternative preferred embodiment, the foetal marker or further foetal marker is glutamine synthase (also known as Glutamate Ammonia Ligase). This enzyme catalyses the production of the bioactive amino acid glutamine by the combination of ammonia with glutamate.

In a further alternative preferred embodiment, the foetal marker or further foetal marker is Ara-70 (also known as Nuclear co-activator 4). This protein belongs to a family of nuclear co-activator transcription factors that in basic terms is involved in regulating the expression of specific genes.

In an additional alternative preferred embodiment, the foetal marker or further foetal marker is Ara-54 (also known as RNF14). Interestingly, like ARA-70, this is another Androgen receptor associated transcription co-activator.

In a still further alternative preferred embodiment, the foetal marker or further foetal marker is human hypothetical protein MGC10526 or MGC10233, FLJ20202, DCN-1 protein, RAB5A, HSP-7C (also known as Heat shock 70 kDa protein 8), EF1A1 (also known as EF-1 alpha-1, elongation factor 1 A-1, eEF1A-1, elongation factor Tu and EF-Tu), GRP78 (also known as immunoglobulin heavy chain-binding protein, BiP, Endoplasmic reticulum lumenal Ca²⁺ binding protein grp 78), MYL4 (also known as myosin light chain alkali GT-1 isoform), DnaJ homolog subfamily B member 14, Vinculin, Desmoplakin, AMMECR1-like protein, Extracellular matrix protein 2 precursor protein, uncharacterised protein Cxorf57, Peroxiredoxin 1 or Peroxiredoxin 2.

The method according to the invention may further comprise a step of separating the selected foetal cells from non-equivalent maternal cells in a sample, this step comprising identifying cells having a different expression pattern of at least one non-foetal marker compared to the expression pattern of the marker in a non-equivalent maternal cell and separating the identified cells from the other cells in the sample. In the case where the selected foetal cells are erythroblasts or other erythroid cells, such a non-foetal marker may be an erythroid specific marker such as glycophorin A, B, C or D, a Rh protein, a Rh-associated protein, Kell glycoprotein. Preferably, the marker is glycophorin A.

The isolated sample of maternal blood may be suitable to be returned to a subject from which it has been obtained. For example, the sample may be part of a line system whereby blood is removed from the mother and subsequently returned, e.g. during an aphaeresis process.

According to a second aspect of the invention, there is provided a method of cultivating foetal cells, the method comprising enriching cells having a different expression pattern of at least one foetal marker compared to the expression pattern of the marker in an equivalent maternal cell, the at least one foetal marker being selected from: HSP-60, a monoamine oxidase, glutamine synthase, Ara-70, Ara-54, human hypothetical proteins MGC10526 or MGC10233, FLJ20202, DCN-1 protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, Peroxiredoxin 2.

Preferably, the foetal cells to be cultivated have been isolated using a method comprising a method according to the first aspect of the invention.

According to a third aspect of the invention, there is provided a cell sample containing isolated cells obtainable or obtained by a method comprising a method according to the first aspect of the invention. Preferably, the cell sample contains cells cultivated by a method according to the second aspect of the invention.

Preferably, the cells of the method according to the first or second aspects of the invention and the sample according to the third aspect of the invention are erythroid cells such as erythroblasts. Presently, it is thought that foetal erythroblasts are present in the maternal circulation at a concentration of one foetal cell to from 1×10⁶ to 1×10⁷ maternal nucleated cells. Other foetal cell types which are present in maternal blood are contemplated such as lymphocytes, mesenchymal stem cells and placentally derived trophoblasts, although erythroblasts are particularly preferred as foetal (Y-chromosome carrying) lymphocytes persist for decades, including into subsequent pregnancies. Furthermore, trophoblasts exhibit chromosomal mosaicism and are rapidly entrapped in maternal lungs due to their large size. Erythroblasts are committed to develop along the erythroid pathway and are unlikely to persist into subsequent pregnancies. They are present at the maternal circulation in relatively high abundance. They are, therefore, suitable cells for use in prenatal diagnoses, since any foetal erythroblasts present in the maternal blood will be derived from the current foetus.

According to a fourth aspect of the invention, there is provided a foetal cell isolation kit, comprising means of detecting whether a cell has a different expression pattern of at least one foetal marker compared to the expression pattern of the marker in an equivalent maternal cell, and means of separating a cell having the different expression pattern of the at least one foetal marker from a cell which does not have the different expression pattern of the at least one foetal marker, characterised in that the foetal marker is selected from: HSP-60, a monoamine oxidase, glutamine synthase, Ara-70, Ara-54, human hypothetical proteins MGC10526 or MGC10233, FLJ20202, DCN-1 protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, Peroxiredoxin 2.

According to a fifth aspect of the invention, there is provided a method of prenatal disease diagnosis, the method comprising the step of obtaining isolated foetal cells by a method comprising a method according to the first aspect of the invention.

Preferably, the method further comprises a step of determining whether the isolated foetal cells contain an indicator of a disease. For example, in the case of Down syndrome, this would be indicated by the presence of an extra copy of chromosome 21 in the foetal cells. Diagnosis of numerous other diseases entails the genetic analysis of known mutations of a particular gene. For example, in diagnosis of Cystic Fibrosis, known mutations in the CTFR gene (e.g. the mutation ΔF508), which are small deletions, gross deletions, or single nucleotide exchanges, would be detected. Such an analysis would be carried out on DNA extracted from the isolated foetal cell, using either a manual or automated procedure for DNA extraction from single or small numbers of cells, such procedures being well known to the skilled person. Extracted DNA may or may not be amplified using a global amplification protocol, for example those used in forensics applications, termed low copy number analysis.

According to a sixth aspect of the invention, there is provided a method of isolating foetal cells from maternal blood, the method comprising identifying cells having a different expression pattern of at least one foetal marker compared to the expression pattern in an equivalent maternal cell and selecting the identified cells, characterised in that the foetal marker is selected from: HSP-60, a monoamine oxidase, glutamine synthase, Ara-70, Ara-54, human hypothetical proteins MGC10526 or MGC10233, FLJ20202, DCN-1 protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, Peroxiredoxin 2.

The method may further comprise a step of separating the selected foetal cells from non-equivalent maternal cells in a sample, this step comprising identifying cells having a different expression pattern of at least one non-foetal marker compared to the expression pattern of the marker in a non-equivalent maternal cell and separating the identified cells from the other cells in the sample. In the case where the selected foetal cells are erythroblasts, such a non-foetal marker may be an erythroid specific marker such as glycophorin A, B, C or D, a Rh protein, a Rh-associated protein, Kell glycoprotein. Preferably, the marker is glycophorin A.

According to a seventh aspect of the invention, there is provided apparatus for use in the method according to the first or sixth aspects of the invention, the apparatus comprising means for detecting whether a cell has a different expression pattern of at least one foetal marker compared to the expression pattern in a maternal cell, and means for separating a cell having the different expression pattern of the at least one foetal marker from a cell which does not have the different expression pattern of the at least one foetal marker, characterised in that the foetal marker is selected from: HSP-60, a monoamine oxidase, glutamine synthase, Ara-70, Ara-54, human hypothetical proteins MGC10526 or MGC10233, FLJ20202, DCN-1 protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, Peroxiredoxin 2. Where a cell is detected which is expressing at least one foetal marker on a surface membrane of the cell and is separated from a cell which is not expressing that marker on a surface membrane, the means of detecting whether a cell is expressing the marker and/or means of separating the cell expressing the marker may take the form of a support comprising an affinity separation material. For example, where the foetal marker is HSP-60, the affinity separation material may be anti-HSP-60 antibody or aptamer.

According to an eighth aspect of the invention, there is provided a method of determining that a cell is a foetal cell, the method comprising detecting in the cell (i.e. within the cell or on the surface of the cell) at least one foetal marker having a different expression pattern compared to the expression pattern in an equivalent maternal cell, characterised in that the foetal marker is selected from: HSP-60, a monoamine oxidase, glutamine synthase, Ara-70, Ara-54, human hypothetical proteins MGC10526 or MGC10233, FLJ20202, DCN-1 protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, Peroxiredoxin 2. Therefore, the method may be used to confirm that a cell is a foetal cell, for example as part of a positive control in cell isolation techniques.

### BRIEF DESCRIPTION OF THE FIGURES

Methods of selecting and separating foetal cells will now be described, by way of example only, with reference to the accompanying FIGS. 1 to 13, in which:

FIG. 1 shows a diagrammatic representation of the type of results of the two-dimensional electrophoresis method used to identify foetal markers from erythroid cells;

FIG. 2 shows representative results of further two-dimensional electrophoresis experiments.

FIG. 2A shows the results of experiments conducted with adult erythrocyte membranes.

FIG. 2B shows the results of experiments with foetal erythroid cell membranes (22 weeks).

FIG. 2C shows the results of experiments with foetal erythroid cell membranes (26 weeks);

FIG. 3 shows the results of the two-dimensional electrophoresis experiments depicted in FIG. 2 in which the position of heat-shock protein 60 is highlighted in the foetal gels. FIG. 3A corresponds to FIG. 2A; FIG. 3B corresponds to FIG. 2B; and FIG. 3C corresponds to FIG. 2C;

FIG. 4 shows the results of DIGE experiments. FIG. 4A shows the results of experiments conducted with adult cells and foetal cells (26 weeks gestation) in which the heat-shock protein 60 foetal specific protein is ringed. FIG. 4B shows the results of experiments with adult cells and foetal cells (22 weeks gestation) in which the heat-shock protein 60 foetal specific protein is again ringed.

FIG. 5 shows analysis screen images from DeCyder software analysis for each of the gels described above. The FIG. 5A screen image is representative of heat-shock protein 60 spot analysis between adult and foetal (22 weeks gestation) samples. The FIG. 5B screen image is representative of heat-shock protein 60 spot analysis between adult and foetal (26 weeks gestation) samples;

FIG. 6 shows the results of Western blot analysis of adult and foetal erythroid cell membranes. FIG. 6A shows a blot of proteins from a cordocentesis sample (26 wks) and 6 adults having varying Rhesus (Rh) phenotype; FIG. 6B shows a blot of proteins from a cordocentesis sample (22 wks) and 6 random adult blood donors; FIG. 6C shows a blot of proteins from a cordocentesis (26 wks), cord (39 wks, term), maternal (15 wks), maternal (15 wks), random adult; FIG. 6D shows a blot of proteins from cord (39 wks, term), random adult, cordocentesis (22 wks), 5 random adult donors; FIG. 6E shows a blot for G3PDH, a housekeeping gene, used to blot all the same samples as a positive control and to control for equal protein loading concentration;

FIG. 7 shows flow cytometry analysis of glycophorin A and heat-shock protein 60 labelled mononuclear cells isolated from adult peripheral blood samples;

FIG. 8 shows flow cytometry analysis of glycophorin A and heat-shock protein 60 labelled mononuclear cells isolated from foetal cord blood samples;

FIG. 9 shows flow cytometry scatter plots of labelled mononuclear cells obtained from adult peripheral blood showing expression profiles of erythroblasts (GPA+) and HSP-60+ mononuclear cells;

FIG. 10 shows flow cytometry scatter plots of labelled mononuclear cells obtained from foetal cord blood showing expression profiles of erythroblasts (GPA+) and HSP-60+ mononuclear cells;

FIG. 11 shows the results of real-time PCR analysis of MAOA and MAOB in placenta, foetal and adult erythroblasts. FIG. 11A shows the results of experiments in relation to MAOA; FIG. 11B shows results of experiments in relation to MAOB;

FIG. 12 shows PDQuest analysis of the three 2D gels in FIG. 2, with the circles showing positions of proteins which were upregulated in foetal cells compared to maternal cells and identified by MALDI-TOF analysis; and

FIG. 13 shows the results of 2D electrophoretic comparison of plasma membrane proteins isolated from foetal and adult cultured erythroblasts, with proteins identified as having a different expression pattern in foetal cells compared with adult cells highlighted.

### EXAMPLES

### Identification of Heat-Shock Protein 60 as being Foetal Cell Surface Specific

HSP-60 was identified as being foetal erythroid cell surface specific by comparison of proteins expressed by foetal erythroid cell membranes and adult erythrocyte membranes.

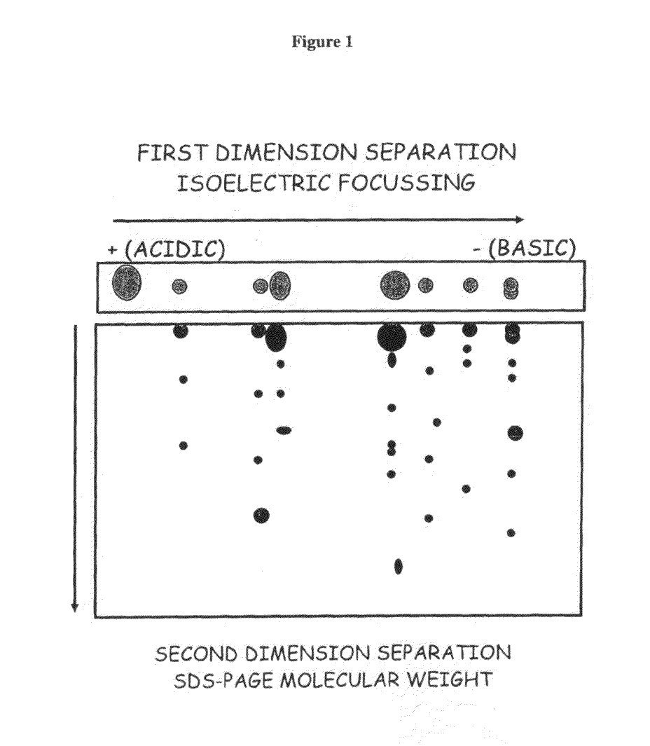

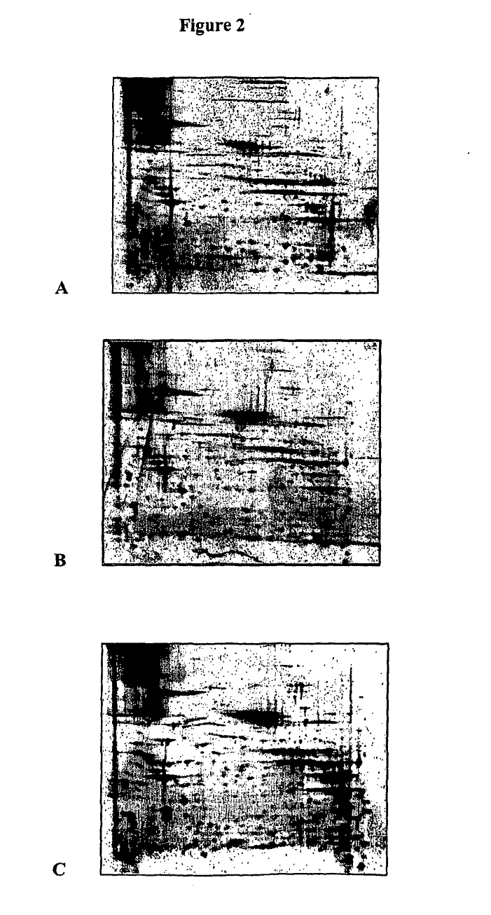

Specifically, red blood cell ghost membranes, prepared and stored at −80° C., were used. After optimisation of membrane solubilisation protocols, a mixture of detergents ASB-14 and CHAPS at concentrations of 0.4% and 1.2% respectively were found to yield the best results. 25 μg of solubilized membranes were used in each two-dimensional electrophoresis experiment. Focusing was achieved by using an immobilised pH gradient and enhanced by adding ampholytes (a mixture of amphoteric species with a range of pI values) to the sample loading buffer. Proteins were loaded at the anode and a current applied to the strip. As the proteins moved towards the cathode they were held in place at the point where their net charge was zero, i.e. at their isoelectric point. The gel strip was then placed in a ready-formed well at the top of a pre-cast SDS gel. Basic SDS-PAGE protocols were then followed allowing the proteins to be separated according to molecular weight, as shown, by way of example, in stylised form in FIG. 1. Gels were stained with Sypro Ruby and scanned on a Typhoon imager and the results are depicted in FIG. 2. A comparison of FIGS. 2A, 2B and 2C shows the similarities and differences between the proteomes of each of the three cell samples.

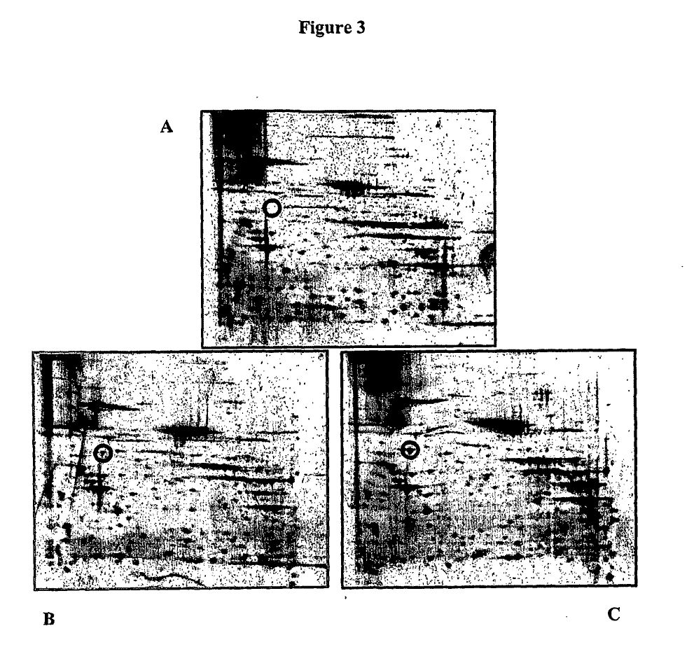

Gel analysis was done using PDQuest software (Bio-Rad) designed to compare two-dimensional gel images and to determine differential protein expression. By accurately land marking proteins for gel alignment, the software determines up- or down-regulation of proteins based on the intensity of protein staining. PDQuest analysis of the three gel images shown in FIG. 2 highlighted proteins both up- and down-regulated between all three gels but, more significantly, found a single protein species present in both foetal gels and absent from the adult gel. After counter staining with Coomassie blue, protein spots were excised from each gel and sent for MALDI analysis. The single protein species present in both foetal and adult gels was identified by MALDI-TOF analysis as HSP-60. The position of HSP-60 in the foetal gel images is highlighted in FIG. 3. It can be seen that no HSP-60 is present in the gel of proteins from the adult membranes shown in FIG. 3A.

### Confirmation of Heat-Shock Protein 60 as Foetal Cell Surface Specific.

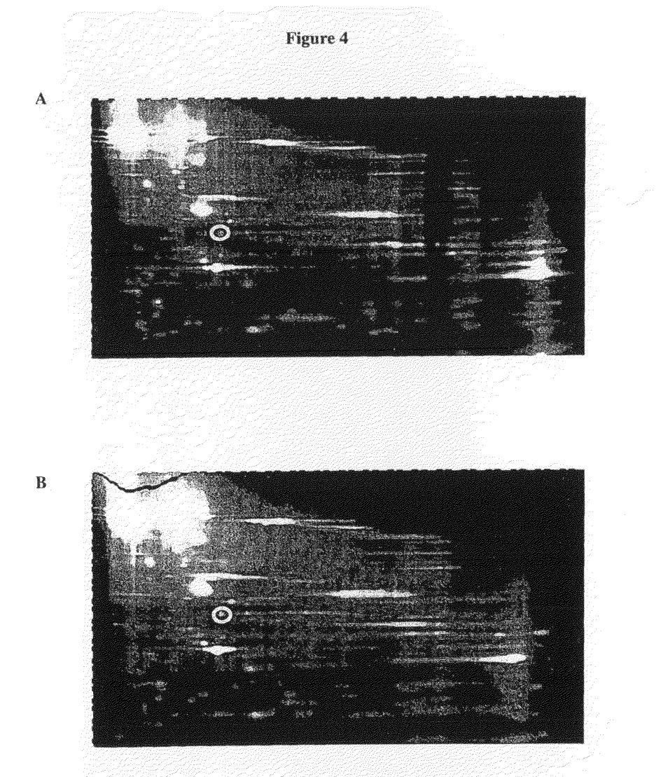

In order to confirm the potential of HSP-60 as a foetal erythroid cell surface specific marker, Differential Gel Electrophoresis (DIGE) analysis was performed on the above samples. DIGE utilises fluorescent dyes to label protein samples before two-dimensional electrophoresis and allows up to three samples to be co-separated and visualised on a single gel. The dyes Cy2, Cy3, and Cy5 are commonly used, each having a different excitation wavelength such that three different scans of the same gel can be performed, each image corresponding to each individual protein sample. The images can then be merged and differences between them determined using image analysis software such as DeCyder (Amersham). As each dye is assured to have a linear response to variations in protein concentration, this technique is quantitative. Reciprocal dying can be employed to ensure that there is no bias in the labelling. The adult cells sample, from R1R1 cells, was run with each foetal sample as shown in FIG. 4. The HSP-60 was again shown to be foetal cell surface specific as highlighted in that Figure.

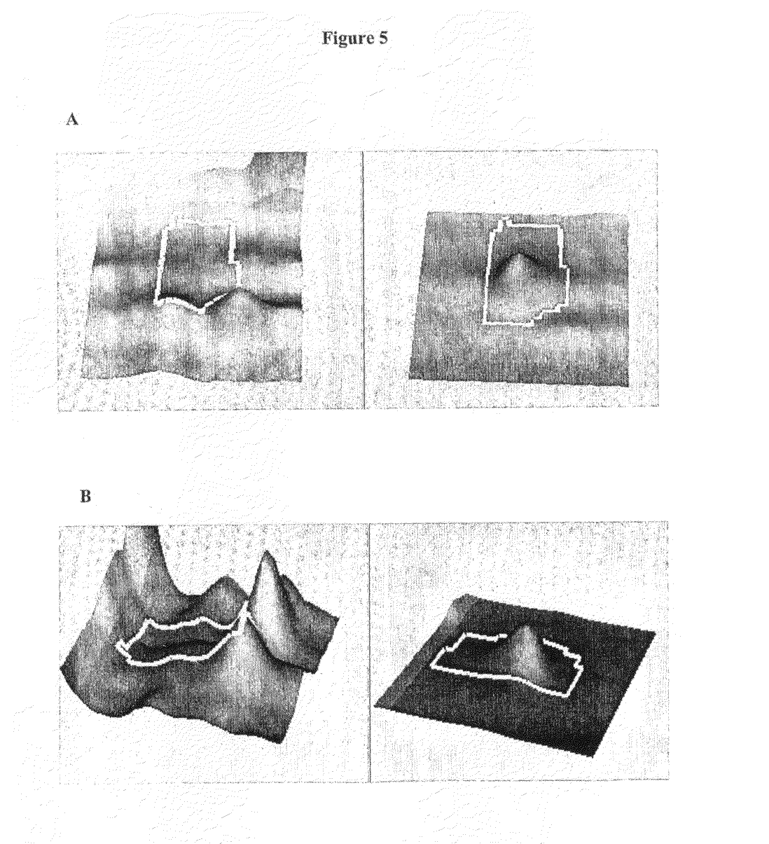

The DeCyder software highlighted many up and down regulated proteins between all three samples during comparison of the three gels. Each gel was analysed alone i.e. the differences between adult foetal gels were determined and then the two gels compared to each other (incorporating differences between the two foetal samples as well). Once again, the spot representing HSP-60 was highlighted as being present in both foetal samples and not in the adult sample. The software analysis screen for HSP-60 in each gel is shown in FIG. 5. Specifically, by comparison of FIGS. 5A and 5B, one can see that the HSP-60 was present only in the membranes from foetal samples (the right hand 3-D image in these Figures).

### Western Blot Analysis of Adult and Foetal Erythroid Cell Membranes to Determine the Presence or Absence of Heat-Shock Protein 60.

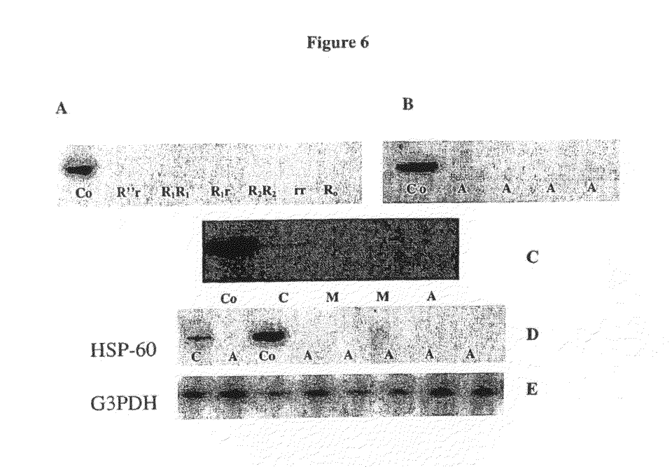

Having confirmed by two techniques, as described above, that HSP-60 was only present in foetal and not in adult erythroid cell membranes, an anti-HSP-60 antibody was purchased from BD Biosciences in order to allow Western blot analysis of this protein in a larger number of samples. Mature erythrocytes were isolated from either adult blood donors or foetal erythroid cells at various stages of gestation. The erythroid cells were then subjected to hypotonic lysis to produce purified membranes (or “ghosts”) and then subjected to SDS-PAGE and Western blot analysis using anti-HSP-60. Erythroid cell membranes were isolated from various sources including random adults, a 26 week foetus, a 39 week foetus (i.e. umbilical cord blood), and maternal adult erythrocyte membranes (15 weeks gestation). FIG. 6 illustrates the complete lack of reactivity of anti-HSP-60 with membranes derived from six adult blood donors and confirms the foetal specificity of this protein. Importantly, HSP-60 appears expressed at a much lower level in the cord samples (term), providing further evidence that surface expression of HSP-60 is foetal specific. At 39 weeks the transition of erythroid cells from foetal to neonatal is occurring and the cells are not in a hypoxic environment.

It has been demonstrated, using fluorescent protein labelling techniques, that foetal erythroblasts generated following the isolation of CD34+ stem cells from cord blood show cell surface expression of HSP-60 (data not shown). Equivalent cells those from adults show intracellular localisation of the protein.

Method for Double Labelling of Erythroblasts with Two Erythroid Markers—Glycophorin A (CD235a) and HSP-60 for Isolation of Foetal Erythroblasts from Maternal Blood Samples for Non-Invasive Prenatal Diagnosis.

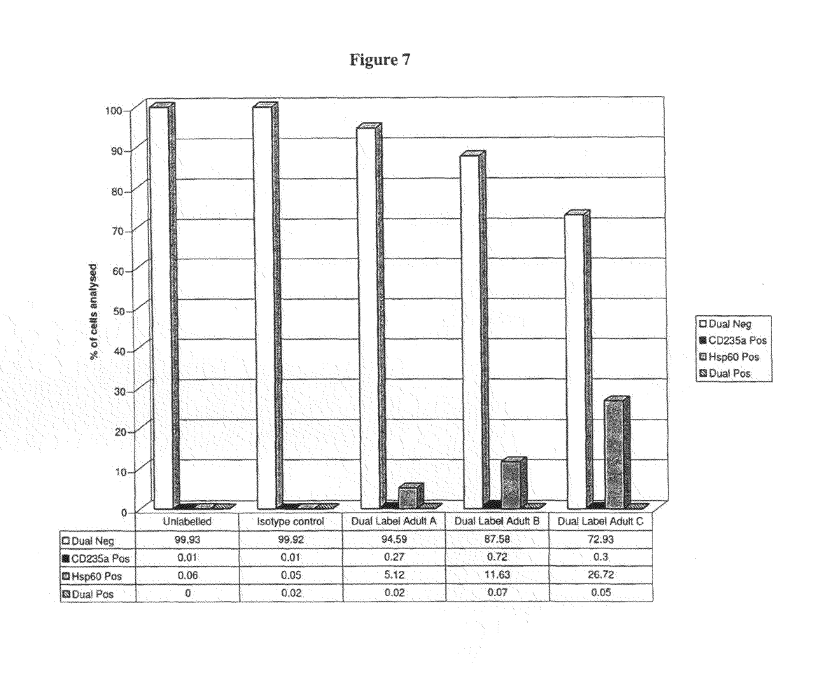

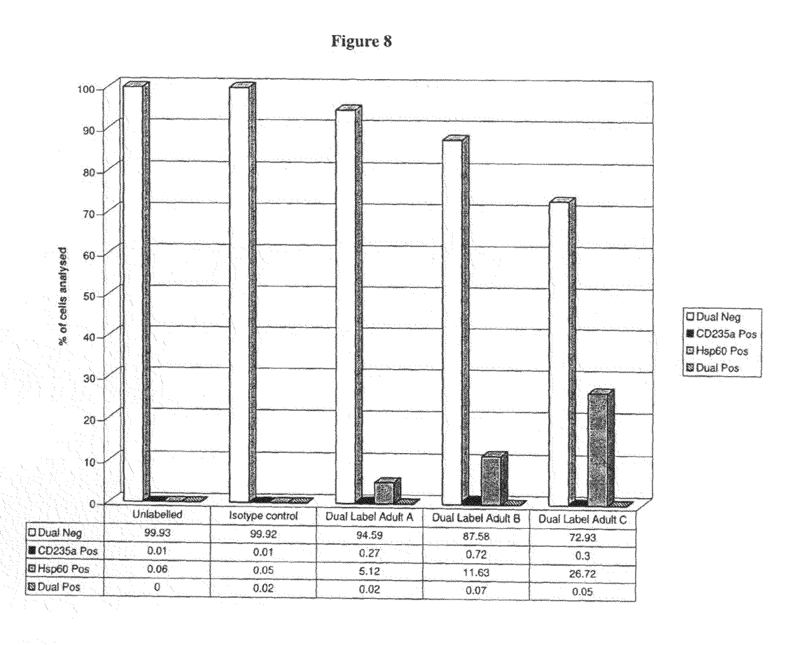

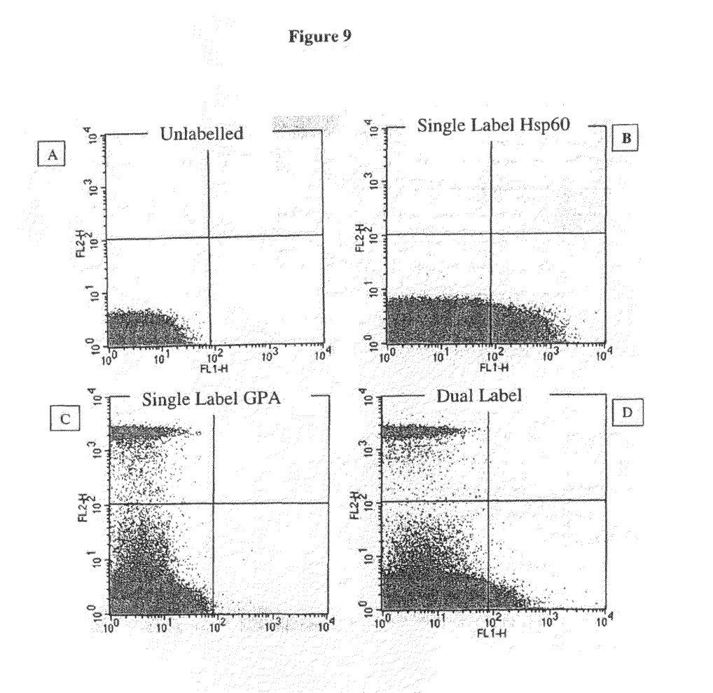

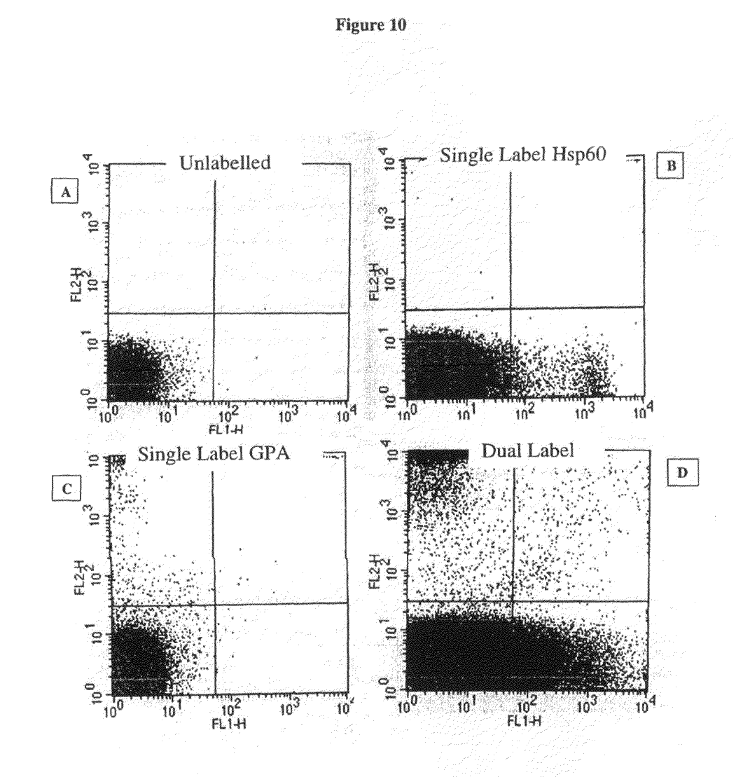

The work outlined above clearly demonstrated that membrane localised HSP-60 is specific for foetal but not adult erythroblasts. However, membrane-localised HSP-60 has been found in a significant proportion of adult mononuclear cells such as leukocytes (from 5 to 26%) (see FIG. 7 and FIG. 9 panel B). Therefore, the inventors developed a method to enrich or purify foetal erythroblasts from a maternal blood sample by elimination of the adult mononuclear HSP-60+ fraction by virtue of the fact that they do not express the erythroid-specific marker glycophorin A (CD235a). Double-positive GPA and HSP-60+ cells are essentially absent in adult samples (see FIGS. 7 and 9), but a small but significant proportion of foetal mononuclear cells (i.e. erythroblasts) present in foetal cord samples are double-positive for HSP-60 and GPA (FIGS. 8 and 10). It is these dual labelled cells that are the specific target cell type for use in non-invasive prenatal diagnosis.

Adult buffy coat samples and Cord Blood (foetal 39 weeks term) samples were processed according to the following protocol:
1) Thirty ml of each sample was added to a Sigma Accuspin histopaque column and centrifuged at 1000×g for 15 min at 18-26° C.
2) The plasma layer was taken off and the mononuclear cell layer transferred to a 50 ml falcon tube.
3) Cells were washed with 25 ml of PBS and pelleted by centrifugation at 2,000 rpm for 10 min at 18-26° C.
4) Cell pellets were resuspended in 25 ml of red blood cell lysis buffer (150 mM ammonium chloride, 130 μM EDTA, 10 mM potassium bicarbonate) and placed on a rocker to facilitate constant mixing for 10 min at room temperature.
5) Cells were pelleted by centrifugation at 2,000 rpm for 10 min.
6) Cells were resuspended in 1 ml of PBS and a cell count performed using a light microscope and a haemocytometer.
7) 1×10⁶ cells from each sample were added to 10 μl of PE conjugated Anti-CD235a (GPA) and 10 μl of FITC conjugated Anti-HSP-60 monoclonal antibodies, in a final volume of 100 μl.
8) Labelling reactions were then placed in the fridge for 30 min.
9) Unbound antibodies were washed off by pelleting the cells at 3,000 rpm in a bench top centrifuge for 5 min and removing the supernatant.
10) Cells were washed twice in 2 ml of PBS.
11) Cells were finally resuspended in 500 μl of PBS and analysed on the FACS machine.
   FL1-H channel=FITC
   FL2-H channel=PE

### Negative Controls:

1) Unlabelled cells
2) Isotype control—Mouse IgG 1 FITC+Mouse IgG 1 RPE

### Antibodies:

### FITC-conjugated Anti-Hsp60 (Isotype Mouse IgG1)

### RPE-conjugated Anti-CD235a (Glycophorin A) (Isotype Mouse IgG1)

FIG. 7 shows three different adult samples and FIG. 8 shows three different foetal samples, with their percentage (gated) expression of both markers. Adult dual-labelled (i.e., having significant levels of GPA and HSP-60) cells show similar patterns to the negative isotype control sample. There are significant levels of expression of HSP-60 on non-erythroid mononuclear cells in adult peripheral blood ranging from 5.12 to 26.72%. Foetal dual-labelled cells show significantly higher levels of expression to the negative isotype control sample. This is consistent with the known higher proportion of circulating erythroblasts in foetal blood.

In FIG. 9, expression patterns of cells carrying GPA were analysed in the FL2 channel, whilst that of HSP-60 in the FL1 channel. Adult erythroblasts can be visualised in the top left quadrant of panels C and D. In FIG. 10, expression patterns of cells carrying GPA were also analysed in the FL2 channel, whilst that of HSP-60 in the FL1 channel. In this Figure, foetal erythroblasts can be visualised in the top left and right quadrants of panels C and D. Significant numbers of foetal erythroblasts (as defined by their expression of GPA) lack the expression of HSP-60 (see panel D, top left-hand quadrant). This corresponds with the strong expression of HSP-60 on foetal erythroid cells during gestation, although expression is present at diminished levels on foetal cord erythroid cell membranes (FIG. 6).

The finding that HSP-60 expression is significantly stronger on erythroid cells during pregnancy, as compared to cord blood (analysed here by flow cytometry) indicates that the dual-labelling approach is a means of isolating of foetal erythroblasts from maternal blood. Purification strategies using cell isolation protocols (Magnetic activated cell sorting, MACS or Flow activated cell sorting, FACS) that use glycophorin A and HSP-60 as ligands therefore lead to enrichment or purification of foetal erythroblasts. These cells can then be used in downstream diagnostic assays to further develop non-invasive prenatal diagnosis using foetal cells as the source of foetal material.

Any surface marker (carbohydrate antigen or erythroid protein) on the surface or cytoplasm of the erythroid cells which is erythroid specific (for example, Rh proteins, Rh associated glycoprotein, glycophorins B, C or D; Kell glycoprotein) can replace the choice of marker coupled with HSP-60.

### Identification of Monoamine Oxidase A (MAOA) and Monoamine Oxidase B (MAOB) as Foetal Specific Enzymes.

The genes encoding the enzymes MAOA and MAOB have been identified as being up-regulated in foetal erythroblasts. The enzymes were then confirmed as being foetal specific by quantitative real-time PCR analysis of adult bone marrow and foetal umbilical cord cDNA.

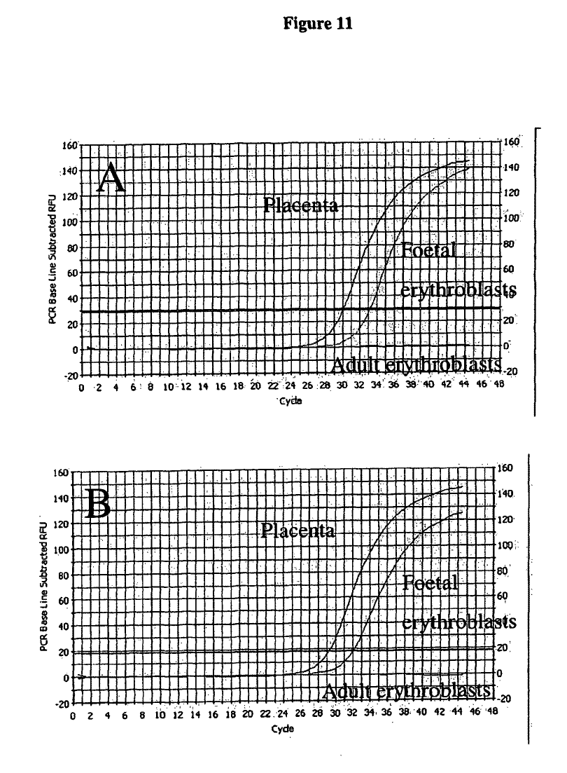

Using MAOA- and MAOB-specific primers, cDNA derived from glycophorin A+ erythroblasts was amplified and detected using SYBR green dye. It is clear from the results shown in FIG. 11 that both MAOA and MAOB mRNA is expressed in erythroblasts isolated from foetal umbilical cord, but not from adult-derived erythroblasts. A positive control (placental cDNA) was included, which is known to express MAOA and MAOB to high levels. The normalisation control glyceraldehyde-3-phosphate hydrogenase (G3PDH) showed no variation in expression between three samples.

The expression of these enzymes in foetal cells could lead to a different and potentially complimentary method of selecting or enriching foetal cells to that described for the foetal cell-surface marker HSP-60 described above. Selective culturing can be used by the use of the well characterised substrates differently metabolised by these enzymes in foetal cells, e.g. serotonin, epinephrine and norepinephrine. MAOA selectively oxidises serotonin and adrenaline; MAOB selectively oxidises phenylethylamine, benzylamine and tyramine; both monoamine oxidases oxidise dopamine. Alternatively, fluorescent markers/probes can be employed in order to allow visualisation of substrate metabolism products produced within the foetal cells only. These substrates will have been converted by the monoamine oxidase present in the foetal cells to a fluorescent product. Such probes have been described recently in the literature (Chen et al. (2005) J. Am. Chem. Soc. 127 4544-4545). This technique would allow a FACS-based approach to be utilised in separation of foetal and maternal cells, whereby monoamine oxidase-expressing foetal erythroblasts producing fluorescent substrates can be separated to homogeneity from the maternal counterparts.

Identification of Various Markers as being Upregulated in Foetal Cells Compared to Maternal Cells.

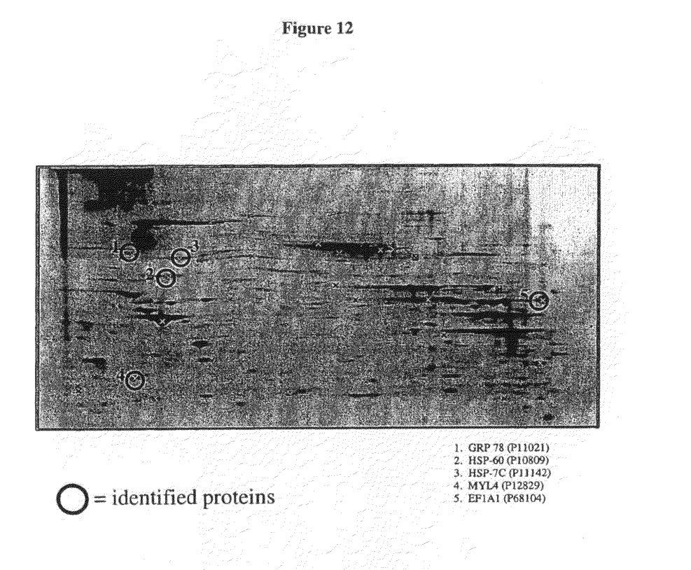

The PDQuest analysis of the three gel images shown in FIG. 2 is shown in FIG. 12. Several proteins were identified as being more highly expressed in foetal membranes than in maternal membranes. As well as HSP-60, discussed above, the other proteins were identified as GRP 78, HSP-7C, MYL4 and EF1A1 (circled).

### Plasma Membrane Protein Analysis of Adult and Foetal Cultured Erythroblasts.

The earliest haemopoietic progenitors possess the cell surface marker CD34. This marker was utilised to isolate stem cells and by exposure to a specific cytokine cocktail, the cells were driven down the erythroid lineage.

Cord blood or adult peripheral blood buffy coats were layered over Histopaque. After centrifugation the samples had separated into an upper plasma layer, an interface layer containing nucleated cells and a lower red cell layer. The interface layer was removed, washed and any remaining red cells lysed. The samples were then magnetically labelled with a biotinylated antibody to CD34 and run through a column in a magnetic field using the MiniMACS system (Direct CD34 Progenitor Cell Isolation Kit, Miltenyi Biotec). The labelled CD34⁺ cells were retained in the magnetic column whilst unlabelled cells were free to flow through. The MiniMACS columns were then removed from the magnetic field and the CD34⁺ cells eluted through the column. The CD34+ cells were cultured in a serum free media (StemSpan, Stem Cell Technologies) supplemented with erythropoietin (3 U/ml), stem cell factor (10 ng/ml), IL-3 (1 ng/ml), low density lipoprotein (40 μg/ml) and FK506/Prograf (0.1 ng/ml). They were maintained at a concentration of 1×10⁵ cells/ml and differentiated through the erythroid pathway from uncommitted stem cell through to erythroblast stage.

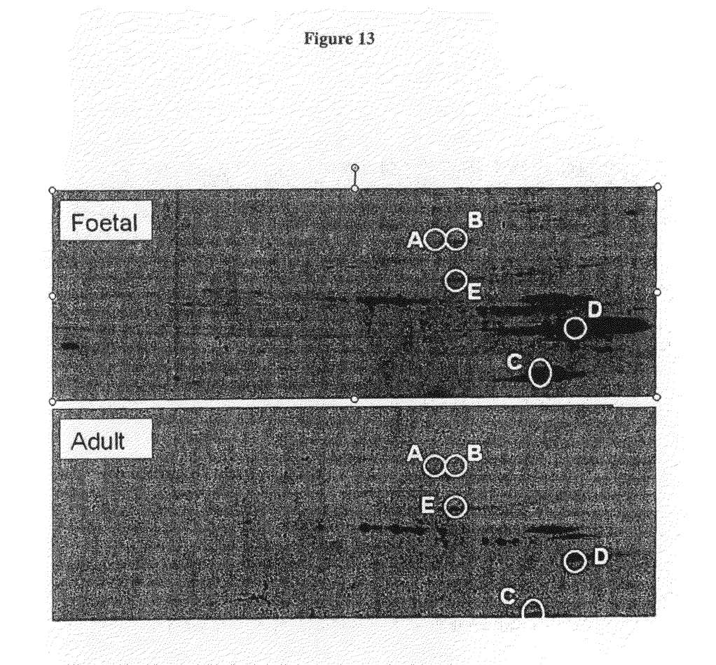

Fractionation of plasma membrane proteins from 1×10⁷ of foetal and adult cultured erythroblasts was performed using the Qproteome Plasma Membrane Protein Kit (QIAGEN) according to the manufacturer's instructions. Isolated proteins were then concentrated by TCA precipitation and 2D gel electrophoresis performed. FIG. 13 shows a magnified area of the resultant Sypro Ruby stained gels.

Clear differences between the number and level of expression of proteins isolated from the foetal and adult samples are apparent. Mass spectrometric analysis enabled the identification of the foetal erythroblast specific proteins Vinculin (Accession P18206, circle A) and DnaJ homolog subfamily B member 14 (Accession Q8TBM8, circle B). The proteins Desmoplakin (Accession P15924, circle C) and AMMECR1-like protein (Accession Q6DCA0, circle D) are shown to be upregulated in foetal cells. Also identified was the Extracellular matrix protein 2 precursor protein (Accession O94769, circle E), shown to be upregulated in adult cells.

Isolation of Foetal Erythroblasts from Maternal Peripheral Blood Using HSP-60 as a Marker

Foetal cell specific markers such as HSP-60 can be used in the isolation of foetal erythroblasts from maternal peripheral blood as set out generally below by way of example:
1. Take a maternal peripheral blood sample (10-20 mL).
2. Perform density centrifugation/red cell lysis to isolate nucleated cells from the maternal peripheral blood sample, using a Histopaque® or Ficoll® density separation medium. Alternatively, using a single step cell isolation procedure from the peripheral blood sample directly by use of a marker according to the invention, no nucleated blood cell enrichment procedure may be required.
3. Perform immunoaffinity isolation of foetal erythroblasts using anti-HSP-60 coated beads.
   3a. Optionally, a preliminary isolation of erythroblasts using (for example) anti-glycophorin A beads can be performed, prior to the use of anti-HSP-60, to isolate erythroblasts (both foetal and maternal) from the maternal peripheral blood sample.
   3b. As an alternative to step 3a, after the use of anti-HSP-60, foetal erythroblasts can be separated from non-erythroblast cells expressing HSP-60 on the cell membrane, by use of, for example, anti-glycophorin A beads.
4. Elute foetal erythroblasts.
5. Using a one step detergent based method, lyse the cells and proceed immediately with single cell genomic DNA amplification using a thermocycling protocol, with or without prior enrichment of DNA using a global amplification protocol.
6. Use this genetic material for example for multiplex ligation-dependent probe analysis (MLPA) analysis of genetic disease markers, quantitative fluorescent PCR analysis, PCR amplification procedures, gene chip, DNA sequence analysis.
   Isolation of Foetal Erythroblasts from Maternal Peripheral Blood Using MAOA or MAOB as a Marker.

Foetal cell specific markers such as MAOA or MAOB can be used in the isolation of foetal erythroblasts from maternal peripheral blood as set out generally below by way of example:
1. Take maternal peripheral blood sample (10-20 mls).
2. Perform density centrifugation/red cell lysis to isolate nucleated cells.
3. Optionally, a preliminary isolation or enrichment of erythroblasts using (for example) anti-glycophorin A magnetic beads can be performed.
4. Incubate erythroblasts with dye which will be transported inside the cells and converted to a fluorescent product by the action of MAOA or MAOB, as appropriate.
5. Sort foetal from adult erythroblasts using a flow activated cell sorter (or other means of separating cells).
6. Using a one step detergent based method, lyse the cells and proceed immediately with single cell genomic DNA amplification using a thermocycling protocol.
7. Use this genetic material for e.g. MLPA analysis of genetic disease markers, PCR amplification procedures, gene chip, DNA sequence analysis.

1. A method of isolating foetal cells from a sample of maternal blood, the method comprising:
identifying cells having a different expression pattern of at least one foetal marker compared to the expression pattern of the marker in an equivalent maternal cell; and
selecting the identified cells, wherein said at least one foetal marker is selected from the group consisting of: HSP-60, a monoamine oxidase, glutamine synthase, Ara-70, Ara-54, FLJ20202, DCN-I protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, and Peroxiredoxin 2.

2. A method according to claim 1 wherein the method comprises identifying cells expressing at least one foetal marker on the cell surface and selecting those cells.

3. A method according to claim 1 or 2 wherein the method comprises selecting cells expressing one foetal marker on the cell surface.

4. A method according to claim 2 further comprising separating the identified cells from cells not expressing the foetal marker on the cell surface.

5. A method according to claim 2 wherein the foetal marker is HSP-60.

6. A method according to claim 1 wherein the at least one foetal marker is a monoamine oxidase.

7. A method according to claim 6 further comprising separating the identified cells from cells not expressing the foetal marker.

8. A method according to claim 2 further comprising the step of:
separating the selected foetal cells from non-equivalent maternal cells having the same expression pattern of the at least one foetal marker.

9. A method according to claim 1 wherein cells are identified which express HSP-60 on the cell surface and an increased amount of at least one foetal marker selected from the group consisting of: a monoamine oxidase, glutamine synthase, Ara-70, Ara-54, FLJ20202, DCN-I protein, RAB5A, HSP-7C, EF1A1, GRP78, MYL4, DnaJ homolog subfamily B member 14, Vinculin, Desmoplakin, AMMECR1-like protein, and uncharacterised protein Cxorf57; or a decreased amount of at least one foetal marker selected from the group consisting of: Extracellular matrix protein 2 precursor protein, Peroxiredoxin 1, and Peroxiredoxin 2.

10. method according to claim 1 wherein cells are identified which express a monoamine oxidase and an increased amount of at least one foetal marker selected from the group consisting of: glutamine synthase, Ara-70, Ara-54, FLJ20202, DCN-I protein, RAB5A, HSP-7C, EF1A1, GRP78, MYL4, DnaJ homolog subfamily B member 14, Vinculin, Desmoplakin, AMMECR1-like protein, and uncharacterised protein Cxorf57; or a decreased amount of at least one foetal marker selected from the group consisting of: Extracellular matrix protein 2 precursor protein, Peroxiredoxin 1, and Peroxiredoxin 2.

11. A method according to claim 1 wherein the foetal marker is a monoamine oxidase.

12. A method according to claim 11 wherein the monoamine oxidase is MAOA.

13. A method according to claim 11 wherein the monoamine oxidase is MAOB.

14. A method according to claim 1 wherein the foetal marker is glutamine synthase.

15. A method according to claim 1 wherein the foetal marker is Ara-70.

16. A method according to claim 1 wherein the foetal marker is Ara-54.

17. A method according to claim 1 wherein the foetal marker is FLJ20202.

18. A method according to claim 1 wherein the foetal marker is DCN-I protein.

19. A method according to claim 1 wherein the foetal marker is RAB5A.

20. A method according to claim 1 wherein the foetal marker is HSP-7C.

21. A method according to claim 1 wherein the foetal marker is EF1A1.

22. A method according to claim 1 wherein the foetal marker is GRP78.

23. A method according to claim 1 wherein the foetal marker is MYL4.

24. A method according to claim 1 wherein the foetal marker is DnaJ homolog subfamily B member 14.

25. A method according to claim 1 wherein the foetal marker is Vinculin.

26. A method according to claim 1 wherein the foetal marker is Desmoplakin.

27. A method according to claim 1 wherein the foetal marker is AMMECR1-like protein.

28. A method according to claim 1 wherein the foetal marker is Extracellular matrix protein 2 precursor protein.

29. A method according to claim 1 wherein the foetal marker is uncharacterised protein Cxorf57.

30. A method according to claim 1 wherein the foetal marker is Peroxiredoxin 1.

31. A method according to claim 1 wherein the foetal marker is Peroxiredoxin 2.

32. A method according to claim 1 wherein the maternal blood is in the form of an isolated sample.

33. A method according to claim 1 wherein the maternal blood is suitable to be returned to a subject from which it has been obtained.

34. A method of isolating foetal cells from maternal blood, the method comprising: identifying cells having a different expression pattern of at least one foetal marker compared to the expression pattern of the marker in an equivalent maternal cell; and
selecting the identified cells, wherein the foetal marker is selected from the group consisting of: HSP-60, a monoamine oxidase, glutamine synthase, Ara-70, Ara-54, FLJ20202, DCN-I protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, and Peroxiredoxin 2.

35. A method of cultivating foetal cells, the method comprising:
enriching cells having a different expression pattern of at least one foetal marker compared to the expression pattern of the marker in an equivalent maternal cell, the foetal marker being selected from the group consisting of: HSP-60, a monoamine oxidase, glutamine synthase, Ara-70, Ara-54, FLJ20202, DCN-I protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised 1 protein Cxorf57, Peroxiredoxin 1, and Peroxiredoxin 2.

36. A method according to claim 35 wherein the foetal cells are isolated after identifying and selecting cells that have the different expression pattern.

37. A cell sample containing isolated cells obtainable by a method comprising a method according to claim 1.

38. A cell sample containing isolated cells obtained by a method comprising a method according to claim 1.

39. (canceled)

40. A method according to claim 1 wherein the cells are erythroblasts.

41. A foetal cell isolation kit, comprising:
means for detecting whether a cell has a different expression pattern of at least one foetal marker compared to the expression pattern of the marker in an equivalent maternal cell, and
means of separating a cell having the different expression pattern of the at least one foetal marker from a cell which does not have the different expression pattern of the at least one foetal marker, wherein the foetal marker is selected from the group consisting of: HSP-60, a monoamine oxidase, glutamine synthase, Ara-70, Ara-54, FLJ20202, DCN-I protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, and Peroxiredoxin 2.

42. (canceled)

43. (canceled)

44. An apparatus comprising:
means for detecting whether a cell has a different expression pattern of at least one foetal marker compared to the expression pattern in a maternal cell, and
means of separating a cell having the different expression pattern of the at least one foetal marker from a cell which does not have the different expression pattern of the at least one foetal marker, wherein the foetal marker is selected from the group consisting of: HSP-60, a monoamine oxidase, glutamine synthase, Ara-70, Ara-54, FLJ20202, DCN-I protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, and Peroxiredoxin 2.

45. A method of determining that a cell is a foetal cell, the method comprising:
detecting in the cell at least one foetal marker having a different expression pattern compared to the expression pattern in an equivalent maternal cell, wherein the foetal marker is selected from the group consisting of: HSP-60, a monoamine oxidase, glutamine synthase, Ara-70, Ara-54, human hypothetical proteins MGC 10526 or MGC 10233, FLJ20202, DCN-I protein, RAB5A, HSP-7C, EF1A1, GRP78, MYL4, DnaJ homolog subfamily B member 14, Vinculin, Desmoplakin, AMMECR1-like protein, Extracellular matrix protein 2 precursor protein, uncharacterised protein Cxorf57, Peroxiredoxin 1, and Peroxiredoxin 2.
